(19) United States
(12) Patent Application Publication
(10) Pub. No.: US 2018/0305638 A1
(43) Pub. Date: Oct. 25, 2018

(54) NON-ALCOHOLIC FRAGRANCES, COMPOSITION AND MANUFACTURE

(71) Applicant: Reimar C Bruening, Freemont, CA (US)

(72) Inventor: Reimar C Bruening, Freemont, CA (US)

(21) Appl. No.: 15/282,510

(22) Filed: Sep. 30, 2016

(51) Int. Cl. C11B 9/02; A61K 8/9789; A61K 8/9794; A61Q 19/00; A61Q 5/00

(52) U.S. Cl. CPC C11B 9/025; A61K 8/9789; A61K 2800/805; A61Q 19/00; A61Q 5/00; A61K 8/9794

(57) ABSTRACT

Fragrance ingredients are described, based on extraction or dilution of natural or synthetic raw materials, or blends thereof, with cosmetic-grade medium-chain hydrocarbon or hydrocarbon-ether solvents (“MCHEs”), comprising C8-C14 chains, straight or branched, of intermediate volatility, for the composition of fragrance concentrates, their manufacture, and embodiments of such concentrates in perfume blends, which can be directly used in household or cosmetic fragrances or for the scenting of personal care preparations, and which can be designated as “non-alcoholic” and—in case of the absence of synthetics—as “all-natural” or “nature-derived”.

This patent relates to Provisional U.S. Patent 62/236,395 [Non-Alcoholic Fragrances, Composition and Manufacture] filed Oct. 2, 2015.

The invention described in this application is not the result of a federally sponsored research or development agreement.

### NON-PATENT CITATIONS

“The next generation of hydrocarbon-based cosmetic fluids”, Ansaldi, A., Cosmetics Business, 47668 (2007).
“PARAFOL Single Cut Paraffins”, Technical Bulletin, SASOL North America, Inc. (2010).
“NEOSSANCE® Hemisqualane”, Sales Bulletin, Centerchem, Inc., (2014).
“Cetiol® Ultimate”, Care Creations™ Sales Bulletin, BASF (2016).
“Cetiol® OE”, Care Creations™ Sales Brochure, BASF (2016).
“Creasil ID GC” Technical Data Sheet CS004; The Innovation Company (

### TECHNICAL FIELD OF THE INVENTION

The present invention relates to fragrance ingredients, fragrance concentrates and perfumes, that are created by high-energy extraction or dilution of natural or synthetic starting materials with solvents composed of nature-derived or synthetic, cosmetic-grade, medium-volatility hydrocarbons or their respective ethers (MCHEs). In particular, the present invention relates to fragrance compositions and perfumes obtained by artful blending of those ingredients without the use of alcohol or water, that can be used directly as non-alcoholic perfumes or in the scenting of skin-care and hair-care products.

### PRIOR ART

Oil-based perfumes and fragrances are the oldest man made scents dating back to at least the times of ancient Egypt and Babylonia. Alcoholic fragrances, on the other hand, have been produced since the Renaissance. Relatively recent are attempts to create novel fragrance carriers other than ethanol. Thus, the creation of non-alcoholic fragrance preparations figures prominently in the current international patent literature.

### REFERENCES CITED

U.S. PATENT DOCUMENTS

| | | |
|---|---|---|
| U.S. Pat. No. 9,119,973 | September 2015 | Warr, J.F. et al. |
| U.S. Pat. No. 8,513,180 | August 2013 | Wiedemann J. et al. |
| U.S. Pat. No. 8,461,099 | June 2013 | Fraser, S. et al. |
| U.S. Pat. No. 8,343,521 | January 2013 | Shick R. et al. |
| U.S. Pat. No. 7,696,147 | April 2010 | Bara I. |
| U.S. Pat. No. 7,425,529 | September 2008 | Souvie M-L. et al. |
| U.S. Pat. No. 7,226,901 | June 2007 | Stora, TH. et al. |
| U.S. Pat. No. 6,869,923B | March 2005 | Cunningham et al. |
| U.S. Pat. No. 6,774,101 | August 2004 | Stora, T. et al. |
| U.S. Pat. No. 6,719,966 | April 2003 | Arbutyn, E.S. |
| U.S. Pat. No. 6,403,109 | June 2002 | Stora, Th. |
| U.S. Pat. No. 6,395,704 | May 2002 | Barnard et al. |
| U.S. Pat. No. 5,871,553 | February 1999 | Spaulding, L |
| U.S. Pat. No. 5,736,505 | April 1998 | Manzo, et al. |
| U.S. Pat. No. 5,585,343 | December 1996 | McGee et al. |
| U.S. Pat. No. 5,468,725 | November 1995 | Guenin E. et al. |
| U.S. Pat. No. 4,301,043 | November 1981 | Sato, H. |

FOREIGN PATENT DOCUMENTS

| | | |
|---|---|---|
| EP | 2786736 | October 2014 |
| | 2719371 (A2) | March 2015 |
| | 2127632 | December 2009 |
| | 1043015 | February 2005 |
| | 1106171A1 | June 2001 |
| CN | 1224687C | October 2005 |
| | 103637942 | March 2015 |
| | 103054756 | April 2013 |
| JP | H08225431 | September 1996 |
| | H08225430 | September 1996 |
| | H08225429 | September 1996 |
| GB | 2398498 | March 2004 |
| WO | 0205771 | January 2002 |

However, none of the recent art cited above describes the use of medium-volatility hydrocarbons or their respective ethers (MCHEs) as fragrance extraction solvents and as fragrance carriers, which lies at the core of the present invention. The prior art falls into three categories: 1) high-molecular weight hydrocarbons are being used as fragrance carriers in mothballs [U.S. Pat. No. 4,301,043], as co-solvents in antiperspirant compositions [U.S. Pat. No. 6,719,966], or as fragrance carriers in scented candles [U.S. Pat. No. 5,871,553]; 2) non-alcoholic fragrances are being produced by emulsification of non-polar fragrance materials into water-based carriers with the help of suitable surfactants [U.S. Pat. No. 8,461,099, U.S. Pat. No. 5,736,505, U.S. Pat. No. 7,226,901, U.S. Pat. Nos. 6,774,101, 5,585,343, U.S. Pat. No. 5,468,725, U.S. Pat. No. 6,403,109, EP2786736, EP1106171, CN103637942, CN103054756, JPH08225431, -30, -29, WO0205771] or by micro encapsulation [U.S. Pat. No. 9,119,973, U.S. Pat. No. 8,513,180]; 3), non-alcoholic fragrances are obtained by using solvents or solubilizers other than alcohol (such as glycols, polyols or siloxanes) to bring non polar fragrance materials into aqueous solutions [U.S. Pat. No. 7,425,529, U.S. Pat. No. 6,395,704, EP2719371(A2), EP2127632, EP1043015, CN1224687C, GB2398498]. No prior art exits that describes the use of MCHE solvents in the creation of non-aqueous, non-alcoholic fragrance materials and their respective perfume blends, which can be directly used on the human skin.

### BACKGROUND OF THE INVENTION

An odor sensation is produced when airborne molecules bind to receptors on the olfactory cilia, which are located on the olfactory bulb inside the human nose. These molecules produce specific neuronal signals that can trigger either negative or positive psychological and behavioral responses. Chemical entities that produce “positive” responses are being defined in the following as “fragrance molecules”, “aroma compounds”, or “odorants”, while fragrant mixtures of those molecules are commonly referred to as “scents”, “fragrance materials”, “fragrances”, “fragrance compositions”, or “perfumes”.

Aroma compounds share certain physical and chemical characteristics:
a), in order to become airborne their molecular weight is normally below 300 Daltons;
b) their vapor pressure must be relatively high in order to allow for volatility;
c) this volatility is associated with low- to intermediate polarities (which minimize the formation of intermolecular hydrogen bonds);
d) the latter property in turn influences their solubility, such that they are more soluble in less polar than in aqueous (more polar) solvent media.

Fragrances, being complex mixtures of aroma molecules, occur widely in nature, for example as botanical or animal scents or cooking aromas, or the scent of a sea shore, to name only a few. Since prehistoric times humans have attempted to capture these scents and use them to enhance their own personas, their food or their environment. This is possible because the fragrant molecules which make up the scents can be extracted from their respective source materials using low-polarity solvents such as oils and fats. For millennia fragrances were thus oil- or fat based extracts of botanical or animal materials, or their artful blends, resulting in perfume oils, unguents or balsams, pomades and fragrant salves. The Arabic culture of the Middle Ages introduced distillation as another method of isolating and at the same time concentrating fragrances from their natural source materials, resulting in what are commonly called essential oils. Those in turn were diluted with oils or fats or added to other extracts to produce ever more sophisticated fragrance blends or perfumes. In this context, oils or fats are called “fragrance carriers” or “carrier solvents”. Ethanol as a fragrance carrier has been in use since the 14th century and has become the solvent of choice in fragrance composition and manufacturing. Advances of synthetic organic chemistry in the 19th century allowed yet for another addition to the palette of fragrance ingredients, namely synthetic aroma molecules, either as the synthetic versions of their natural counterparts, or as completely novel entities with hitherto unknown olfactory characteristics. Today synthetic aroma molecules are the bulk ingredients in most fragrance compositions. Similar to fragrance molecules of natural origin, these synthetics are characterized by relatively low molecular weights, high volatility and low polarity. Consequently, oils and ethanol are still the most widely used carrier solvents in perfumery.

Ethanol based fragrance compositions are widely used as concentrated perfumes (French: “Extraits”) at around 20% concentration of fragrance, or their diluted counterparts, Eau de Cologne (at 5-10%), Eau de Toilette (<15%), or Eau de Partum (>15%), or as scenting components of cosmetic preparations such as creams, body lotions, shampoos and body washes, or in the scenting of household and industrial products and room sprays. However, consumer preferences in some segments of the fragrance and cosmetics markets are trending away from alcohol as a fragrance carrier solvent. The demand for non-alcoholic fragrance is based on perceived drying or irritating effects of alcohol on the skin, on the fact that the alcohol is mostly of synthetic origin (see the demand for natural ingredients below), and—especially in the growing Middle-Eastern market—for cultural reasons. One alternative to alcohol is the use of natural oils. However, oils have certain disadvantages as fragrance carriers, most notably their limited chemical stability and oily skin-feel. The natural instability of plant-based oils poses a problem, since gradual decomposition leads to malodorous components (“rancidity”) which can destroy a scent impression, and thus severely limit product shelf-life. Moreover, oils as fragrance carriers create an “oily” skin-feel that is often disliked by the consumer for esthetic or medical reasons (in cases of acne-prone skin, for example). An additional disadvantage of carrier oils is that they will stay on the skin for considerable time and thus influence the evaporation behavior of the fragrance components. The result is a weaker scent sensation compared to alcoholic perfumes, a fact that limits universal consumer appeal. Finally, the same lack of volatility can lead to staining of fabrics and clothing.

International consumer preferences are also trending towards all-natural and sustainably-produced ingredients, in food as well as in cosmetic or fragrance applications. In the perception of the general public, “natural” or “nature-derived” ingredients rank better than synthetics in terms of potential health and safety concerns. Also, natural materials, especially when sustainably produced, are perceived to leave a lesser carbon footprint than synthetics, which are mostly derived from petrochemicals. Consequently, products that can be designated “natural”, “organic” or “nature-derived” can demand much higher retail pricing than conventional products, and thus the potential for fraudulent designations, descriptions and product labeling becomes obvious. As a result, several consumer protection organizations have sprung up in Europe (ECOCERT, COSMOS, etc.) and the US (USDA, NPA, etc.) which monitor “natural” or “organic” product label claims, provide product certifications in accordance with their published standards, and have issued lists, which designate ingredients that can be called “natural”, “nature-derived”, or “organic”. Products which conform to one of those standards gain an instant and significant market advantage.

In contrast to the manufacturing of synthetic aroma chemicals with its predictable and defined process costs, the cost-effective production of natural fragrance materials poses unique challenges: 1) due to their mostly botanical origin, these materials may be produced in remote geographic areas, can be highly seasonal, and their quality often varies under climatic influences. 2) natural fragrance materials often evoke a “weaker” smell sensation than synthetic materials. This has first and foremost to do with the fact that natural raw materials and ingredients are usually complex mixtures containing hundreds of different compounds of which only a certain percentage contribute to the fragrance. 3) many of the non-fragrant components are highly colored or bear other undesirable characteristics, such as lower solubilities and stickiness, making their use in fine fragrance or cosmetic applications a challenge. When compared to synthetic fragrance materials, nature-derived ingredients are more expensive to procure, and may require elaborate refining processes with profound consequences on production costs and odor profiles. The current industry conundrum is that synthetic materials, while more cost effective, are being increasingly rejected by the consumer, and yet the corresponding refined natural alternatives are driving prices up and thus again limiting customer appeal.

The present invention introduces new fragrance carrier solvents which are comprised of medium-chain (C8-C14) straight or branched hydrocarbons, or their respective ethers, (MCHEs) or blends thereof, which are able to selectively dissolve aroma molecules from natural matrices or pre-extracted raw materials, leaving non-fragrant compounds behind. The resulting solutions are selectively enriched for aroma molecules, have high olfactory impact, and offer production economy, compared to current art. In the following text these solvents will be abbreviated as MCHE.

Considering the limitations of alcohol and oils in the current art of perfume and fragrance production, the more ideal MCHE fragrance carriers, presented here as the essential part of the invention, have the following attributes:
selective solubilization of fragrant components, allowing for their selective extraction and concentration from raw natural materials or synthetic reaction mixtures, while leaving non-scented, strongly colored, and otherwise undesirable compounds behind to be easily separated.
chemical stability and inability to interact with fragrance components in any way that could negatively influence the composition.
sufficient volatility to allow the fragrance to evaporate from the skin as intended and not leave any residue behind that would create either an un-desirable skin-feel or stains on clothing and fabric.
natural origin allowing for certification by ECOCERT, COSMOS, NPA, or any other certifying body, thus enabling the term “natural” or “nature-derived” to appear on product labels and advertising.
chemical inertness and consequently excellent consumer safety in the areas of intended application.

### SUMMARY OF THE INVENTION

The present invention describes fragrance materials, which
a) are based on medium-chain hydrocarbons or their respective ethers (MCHEs) as extraction and/or carrier solvents, preferably of natural origin, medium volatility, and certified as safe for use in leave-on perfumes, and personal care products;
b) can be produced by extraction of raw natural materials such as flowers, leaves, and roots, etc., or of pre-extracted fragrant materials comprising concretes, absolutes, resinoids, and tinctures, known to the art, or synthetic fragrance materials, or mixtures of the above, with said MCHEs;
c) provide the practitioner with solutions that are enriched for fragrant components, (which are naturally present in those raw or pre-extracted materials at higher dilution), are lightly colored and mostly free of non-fragrant ballast matter;
d) can be blended with the same materials from different vintages or provenances in such proportions as to guarantee reproducible quality over extended marketing periods;
e) can be directly used in the creation of complex fragrance blends, concentrates and perfumes without the necessity for further dilution.

In a preferred embodiment, these ingredients are blended into all-natural, non-alcoholic and non-aqueous fragrance concentrates that have stability and esthetic qualities similar to classical alcohol-based perfumes, but improved skin feel.

In another embodiment, conventional fragrance concentrates are treated with aforementioned MCHEs or their blends by means of a high-energy extraction process to produce fragrance concentrates or perfumes with novel and original olfactory characteristics, compared to the starting materials, and yet with the same favorable qualities as described above.

In yet another aspect of the invention, such concentrated fragrance blends—due to their light color and other favorable physical and esthetic characteristics—can be directly incorporated as scenting agents or fragrant emollients into cosmetic, skin-care and hair-care products, such as body mists, creams, lotions, wash-gels, or shampoos, that can be labeled “all natural” or “nature-derived” (if none of the other components in the formula are of synthetic origin).

### DETAILED DESCRIPTION OF THE INVENTION

### Natural Fragrance Materials

A natural fragrance material as defined hereinafter is an essential component of the invention. Natural fragrance materials according to the invention are extracts or dilutions of natural source materials, such as flowers or flower petals (e.g. jasmine, rose, tuberose, etc.), leaves or herbs (e.g. lavender, mint, pine needles, etc.), spice seeds (e.g. cumin, fennel. etc.), barks (e.g. cinnamon), roots (e.g. vetiver), fruits (e.g. vanilla, peach, apricot, etc.), and resins and gums (e.g. benzoin, labdanum, etc.), to name only a few. They can also be obtained by extraction or dilution of pre-extracted materials known to the art as “essential oils” (distillates of various plant materials), “concretes” (extracts of various plant or animal materials with volatile solvents, followed by a concentration step), “absolutes” (extracts of concretes with ethanol, followed by a concentration step, “resinoids” (evaporated extracts of exudates, resins, gums, etc.), and “tinctures” (liquid alcoholic extracts of a fixed solvent/matter ratio), or their respective blends. Traditionally, the solvents used for the production of these materials are low-boiling, and highly flammable petrol fractions such as n-Hexane or petrol ether, alcohol, or liquified carbon dioxide (supercritical fluid extraction, SFE). In contrast, the natural fragrance materials comprising this invention, are based on MCHEs as extractants and carrier solvents, with higher flash-points and hence safer processing qualities, and with esthetic and biological-safety characteristics that allow their direct incorporation in cosmetic skin- and hair-care formulas.

Preferably but not limited to, these MCHE solvents are of natural origin, for example derived from coconut-oil or palm-oil (INCI=International Nomanclature for Cosmetic Ingredients: “Coconut Hydrocarbons” or “Palm Oil Hydrocarbons”). These MCHEs have only recently become commercially available as cosmetic skin emollients under trade names such as “Parafol®” (SASOL Inc., Germany), “Cetiol® Ultimate” (BASF-COGNIS Inc., Germany), both derived from coconut oil and palm oil, and “Neossance®” (INCI=“Hemisqualane”) produced by biotechnological fermentation of corn-derived sucrose by certain microorganisms (Amyris Inc., USA; Centerchem, Inc., USA). Alternatively, synthetic materials with the desired physical characteristics may be used, for example “Isododecane” (INCI), available under the name “Creasil® ID CG” (CoastSouthwest, USA), among many others (refer to “Non-Patent Citations”, above, for the corresponding technical brochures).

The corresponding ethers of the general formula R₂O have the structure (CₙH₂ₙ₊₁)₂O, where n can be any number from 7 to 12, with both hydrocarbon ends R being either identical or different, straight or branched, and which are preferably of natural origin, such as “Octane, 1,1′-oxybis-”, commonly known as “Dicaprylyl Ether” from palm oil (Cetiol® OE, BASF, Germany). As already mentioned, the intended uses for these materials are in the cosmetic and skin care areas, and many have been ECOCERT-certified, since their production processes conform with the certification standards for natural cosmetics.

The preferred MCHE products for this invention, are being marketed either as pure compounds (e.g. n-Dodecane, which is being commercialized under the name of Parafol®12-97) or Hemisqualane (Neossance®), or blends such as Cetiol® Ultimate which consists of n-Undecane (C-11) and n-Tridecane (C-13), or ethers such as Cetiol® OE (see above).

According to the invention, these MCHE liquids are especially useful as fragrance extractants or fragrance carriers, because of their low polarity and concomitant solubilizing selectivity for low-molecular weight fragrance molecules, their volatility at skin temperature, and their superior skin-feel and non-irritant property. Additionally, fragrance ingredients or their respective blends, which are based on these solvents, have a viscosity similar to ethanol and substantially lower than vegetable oils, making them suitable for use in sprays and atomizers.

The invention employs these MCHEs as solvents for the processing of conventional natural or synthetic fragrant materials—either raw or pre-extracted (see above)—or their mixtures, by means of dilution, extraction or maceration (that is a form of static or dynamic extraction at a given temperature over an extended period of time). Unlike conventional aroma extracts with short-chain hydrocarbons of petrochemical origin, such as n-hexane or petrol ether, which have to be removed by evaporation before further use (among other reasons because of their flammability), the claimed non-alcoholic, MCHE-based fragrance concentrates, being of considerably lower flammability, non-irritant and only slightly colored and with fewer non-fragrant contaminants, can be employed directly in fragrance or cosmetic formulations without further dilution or adjustments. Alternatively, MCHE extracts described above may be further concentrated by methods comprising evaporation or partition with diluted natural ethanol, resulting in highly concentrated fragrance materials which are equivalent to what is known to the art as “concretes” or “absolutes”, respectively, with the important difference that these materials are entirely of natural origin and can be declared and certified as such.

In traditional perfumery the concentrated and often deeply colored extracts of natural materials are first blended as such, that is undiluted, then diluted with 70% ethanol (“perfumer's alcohol”), and frozen for an extended period of time to precipitate out most of the colored and non-fragrant by products. In contrast, the present invention allows direct composing and blending of highly fragrant MCHE concentrates into finished perfumes without the necessity for further dilution or time-consuming clarification. In other words, the present invention allows for a new and efficient way to create fragrance blends and perfumes, that can be either employed directly on the human skin or become the fragrant part of a personal care or household formula.

As described above, raw starting materials employed by the invention can be fresh or dried plant or animal parts, or pre-extracted botanical or animal materials, or solvent extracts of those raw materials. Most often, these solvent extracts have physical properties (low solubility, high viscosity/stickiness, deep color, etc.), that make them difficult for direct use in fragrance compositions, perfumery, or in cosmetic formulations. Moreover, only a certain fraction of the mass of these extraction products comprises the actual fragrant molecules and often large batch-to-batch variability can occur. The latter problem stems from factors such as lack of standardization protocols in the countries of origin, or climatic variability during growing and harvest seasons, or variable quality of extraction solvents. Thus, for industrial producers of fragrance materials the creation of products of consistent quality over long sales periods is a major challenge. In this embodiment of the invention, raw materials are treated with MCHE solvents at a pre-determined ratio. Because of their low polarities, which closely match those of most fragrance molecules, the solvents introduced by the invention selectively extract and concentrate aroma compounds into lightly colored, highly-fragrant, low-viscosity concentrates of consistent and reproducible quality, leaving most non-fragrant ballast behind. As it is common in food and beverage production, artful blending of different batches of these concentrates, or batches of different provenances or vintages, allows for an easy and cost-effective way to maintain consistent product quality over extended periods of time.

Similarly, production processes for synthetic fragrance materials go through stages of purification of the raw reaction mixtures. In a related embodiment of the invention, aforementioned MCHE solvents can be used to selectively extract fragrant species from pre-purified reaction mixtures (from which potentially interfering reagents and solvents have been removed) to give highly fragrant concentrates. In the latter, the individual fragrant molecules (e.g. diastereomers or enantiomers) exist at fixed relative concentrations due to their respective solubilities, resulting in high batch-to-batch consistency and reproducibility.

The following are non-limiting examples of suitable processes for the manufacture of fragrance ingredients according to the present invention.

### Example 1

1 kg of freshly picked jasmine flowers is equally divided into 4 500 mL high-density polyethylene centrifuge containers with screw-cap closures (Nalgene, Inc.) and each brought to 400.0 g by addition of the required amount of Parafol® 12-97 (INCI: “Coconut Dodecane”). The bottles are sealed and placed onto a laboratory bottle roller (Thermo Scientific) and rotated for 6 hours at low speed. Afterwards the bottles are placed into a suitable centrifuge (Fisher Thermo) and spun at 500 rpm for 10 minutes. The supernatants are decanted, combined and brought to the desired weight by addition of fresh solvent. This extract is again used for the next batch of 1 kg fresh flowers. After 5 iterations of the extraction/centrifugation sequence, 5 kg of jasmine flowers yield approximately 600 grams of a light-yellow clear extract with a strong true-to-nature jasmine scent (approx. 80% yield). This extract is placed in a suitable container over 50 g of drying agent (e.g. Drierite®, W. A. Hammond DRIERITE Co. LTD), kept in a refrigerator for 12 hours, filtered, and stored in closed containers in a cool and dark place, preferably under a blanket of inert gas. This liquid can be used directly in further blending, the scenting of cosmetic preparations, or as a spray mist.

### Example 2

1 kg of frankincense gum from Oman (Royal Green Hojari; www.arabiandelights.com) is placed in a 2 L blender together with 1 L of Cetiol® Ultimate (INCI: Palm Undecane/Tridecane) and extracted at high speed for 10 minutes. Care has to be taken to keep the temperature at or below 30° C. in order to prevent the gum from becoming sticky. The resulting suspension is filtered through a Büchner funnel under vacuum, and the filtrate (“first extract”) is brought to 1 L by adding fresh solvent. This “first extract” is used to process another 1 kg of gum. Concomitantly, the remaining filtration residues are re-extracted with 1 L of fresh solvent and again filtered off, resulting in a “second extract”. After processing 2 kg of raw material, the filtrates are combined and placed in a refrigerator for 24 hours, at which time additional non-fragrant ballast materials precipitate out. After subsequent filtration 3.5 L of a slightly yellow clear liquid with a strong and true-to-nature incense smell are obtained (87.5% yield). Unlike traditionally produced frankincense resinoids, this liquid does not leave any sticky residue on the skin and can be used directly in further blending and scenting applications.

### Non-Alcoholic Fragrance Compositions and Perfumes

A non-alcoholic non-aqueous fragrance composition or perfume as detailed hereinafter is a further embodiment of the invention.

The perfume composition according to the invention comprises either blends of the aforementioned fragrance ingredients with MCHEs as carrier solvents, either as such or as a starting point for further addition of fragrance materials other than those described above (for example essential oils or synthetic aroma materials). Alternatively, however, the invention describes perfumes which are obtained by extraction or dilution of blends of natural raw ingredients, synthetic raw ingredients, or blends thereof, as they are routinely produced as fragrance concentrates in common perfumery, before being diluted with ethanol to the desired strength of the commercial product (see BACKGROUND, above). In this embodiment of the invention, the aforementioned medium-chain hydrocarbons or ethers (MCHEs) are used as replacement of ethanol in the final step. However, due to their entirely different chemical nature, these solvents are not merely diluents, but, in contrast to ethanol, selectively dissolve the low-polarity fragrant materials from the initial blend and at different ratios compared to the original concentrate. The MCHE solvents at the core of this invention are non-polar in nature. This means that they have a natural affinity to fragrance molecules of low polarity, easily dissolving and thereby separating them from more polar components in the starting mixtures. This phenomenon is observed when, for example, blends of essential oils and other fragrant but often deeply colored plant extracts are being diluted by Parafol® 12-97, Cetiol® Ultimate or Hemisqalane (Neossance®): The blend turns milky and subsequently separates into two layers. The upper layer contains most of the fragrant components but little color, while the lower layer contains mainly more polar and highly colored side products but also more polar and desirable fragrant molecules. According to the present invention, the partition of those more-polar fragrant molecules of the original blend into the upper hydrocarbon layer can be maximized by treating the two-phase system with ultrasound or a high-speed homogenizer for several minutes, followed by storage at near-freezing temperature to allow for a clear separation of the mixture into two layers. The resulting upper (less polar) layer is highly fragrant yet clear and, in contrast to the original blend, almost colorless, which provides an important esthetic advantage when aiming at the scenting of cosmetic formulations. Moreover, the resulting fragrance profile of the upper layer is significantly different from the original untreated blend: it appears to be lighter, more radiant, and overall more “elegant”, which is consistent with the observation that the dilution/extraction with the MCHE carrier seems to concentrate the so-called top-notes (usually the components with the highest vapor pressure and/or the lowest detectability threshold), while extracting the other fragrance molecules in relation to their relative polarities. Under these circumstances, the dissolved low-polarity compounds themselves can act as co-solvents for other fragrant components in the original blend. This complex interaction between solvent and solute (MCHEs and low-polarity aroma compounds) results in fragrance blends where the individual fragrant components are present at different concentrations and proportions than in the original blend. The novelty of this approach lies in the fact that the carrier solvent not only acts as a diluent but actively participates in the selective extraction of fragrance molecules resulting in an entirely novel composition and scent profile. The inventor also observed that these novel perfume compositions can be easily atomized using common pump-sprays, and upon contact with the human skin produce a pleasant, velvety feel; they evaporate in relatively short time due to elevated skin temperature, leaving behind the pure scent on the skin for well over twelve hours. In other words, they behave like classical alcohol- or oil-based perfumes, but without the typical alcohol “sting”, or oily skin-feel.

The following is a non-limiting example of a novel all-natural and non-alcoholic perfume composition according to the present invention.

### Example 3

A blend of natural ingredients (liquids) is created with the aim at an all-natural jasmine-chocolate accord (starting blend in grams):

| ingredient Name | Weight (g) |
|---|---|
| Jasminum auriculatum hydrodistillate | 1.70 |
| (a.k.a. “Ruh Juhi”; White Lotus Aromatics LLC) | |
| Coconut CO2 extract (White lotus Aromatics LLC), | 0.80 |
| Benzoin “Heart” (Robertet) | 0.70 |
| Grapefruit “Ruby” essential oil (White Lotus Aromatics LLC) | 0.50 |
| Vanilla bean extract, certified organic (California Cosmetic | 0.50 |
| Creations) | |
| Cacao absolute “France” (The Perfumery) | 0.50 |
| Cistus absolute (Albert Vieille S.A.) | 0.40 |
| Musk ambrette seed CO2 (The Perfumery) | 0.35 |
| Carrot seed essential oil (Robertet) | 0.03 |
| Ambrettolide natural (Penta) | 0.02 |
| Total concentrate | 5.50 |

This untreated blend is a dark-brown syrup with a strong jasmine/fruity-citrus smell. Two batches of 5.50 grams each were prepared for comparison. To the first batch were added 22 grams of 70% ethanol to bring the concentration of fragrant materials to 20%, which is the common concentration of a classic French perfume. The mixture was shaken for ten minutes, refrigerated for 1 week and cold-filtered to give an alcoholic perfume (“A”) of deep yellow-brown color.

22 g of Parafol® 12-97 (INCI:“Coconut Hydrocarbon) were added to the second batch of the fragrance concentrate (5.50 grams) to again bring the fragrant materials to 20% (w/w).

The cloudy two-phase mixture was submersed into an ultrasound water bath set at “high” (Branson Model 1210) for 10 minutes at room temperature, resulting in a milky liquid. After chilling over night at −10° C. and subsequent thawing of the frozen hydrocarbon, the clear upper phase was aspirated to give the non-alcoholic perfume “B”.

The two perfume blends differ considerably in their physical and sensory qualities:

| | | | Duration | |
|---|---|---|---|---|
| | Color | Scent Description | on Skin | Skin Feel |
| “A” | brown | sweet jasmine, heavy | 24 hours | slightly sticky |
| “B” | light yellow | dry jasmine-chocolate | 24 hours | dry, velvety |

In comparison to “A”, the non-alcoholic perfume “B” exhibited a strong yet harmonious chocolate-jasmine aroma that can be described as dryer, less fruity than “A”. Sprayed onto skin with an atomizer the blend spread easily and with a comfortable warming sensation. After less than a minute there was no residue left, save for the strong yet very elegant fragrance. After 5 minutes on the skin a citrusy amber note developed, which evolved into a very feminine muskiness after an additional 30 minutes. After 3, 12 and 24 hours, the jasmine-musk theme was still clearly dominant with the chocolate notes becoming increasingly noticeable.

Most interestingly, a panel of 6 volunteers rated the non-alcoholic perfume version of the invention unanimously as much too strong compared to the alcoholic version. Only after further diluting the blend to 7.5% did the panel feel a strength equivalence to the alcoholic version (at 20%). Thus it could be demonstrated that the hydrocarbon-based perfume had changed the original scent profile towards a brighter onset that warranted further dilution, giving more integration and elegance, and yet upon skin application behaved just like a classical alcoholic fragrance in the sense of olfactory progression from top-through heart-note and finally dry-down, in a development time frame that is well within the range for any high-class perfume.

It can thus be deducted, that yet another aspect of the invention is the improved economy of perfume production due to the fact that the methodology described produces highly enriched fragrance concentrates compared to methods currently in use. These intermediate concentrates can be significantly diluted with the MCHE solvents detailed above to produce perfumes of odor-strength and quality that are comparable to currently available alcoholic perfumes.

It can therefore be concluded that the perfume materials of the present invention comprise a new composition of matter, in that they substantially differ from traditionally produced perfumes in terms of:
new fragrance profiles from classical starting materials, minimal color (non-staining), and superior skin-feel (non-alcoholic, non-oily, non-sting, non-irritant),
improved production economy due to the selective enrichment of fragrant molecules in the end product,
chemical stability and substantially increased shelf-life in comparison to oil-based fragrances,
the potential for certification as “all-natural”, since the carriers can be of plant origin, such as the MCHEs used herein.

What is being claimed:

1. A method of producing non-alcoholic, non-aqueous fragrance materials, fragrance compositions, or perfumes, with enhanced olfactory and esthetic characteristics, and which may be used either in the scenting of products comprising household products or room sprays, or applied directly to the human skin for uses comprising perfumes or scented emollients, either by themselves or as part of personal care products,
a) wherein said fragrance materials or fragrance compositions comprise extracts of fragrance raw materials of botanical, animal, mineral, or synthetic origin, or
b) wherein said fragrance materials or fragrance compositions comprise extracts of prefabricated fragrance materials known to the art as “concretes”, “absolutes”, “resinoids”, “tinctures”, “macerates”, or “essential oils”, and
c) wherein the materials described in claims 1a and 1b are produced by extraction with solvents comprising medium-chain (C₈-C₁₄) hydrocarbons, straight or branched, preferably of cosmetic grade, and of natural origin, such as “Coconut Hydrocarbons” (INGO, “Hemisqualane” (INCI), or
d) wherein the materials described in claim 1a are produced by extraction with solvents comprising medium-chain hydrocarbon ethers of the general formula (R₂O) having the structure (CₙH₂ₙ₊₁)₂O, where n can be any number from 7 to 12, and where the hydrocarbon ends (R) are either identical or different, straight or branched, preferably of cosmetic grade, and of natural origin, such as, “Dicaprylyl Ether” (INCI), or
e) wherein the materials described in claim 1a are produced by extraction with solvents comprising blends of those solvents claimed in claims 1c and 1d, with or without additional solvents, that are known to the art for extraction purposes, and preferably of cosmetic grade and of natural origin, and
f) whereby the blends claimed in claim 1e are composed in such a way as to specifically extract or dissolve desirable fragrant components from complex mixtures based on their respective polarities and solubilities;
g) wherein the extraction of materials described in claim 1a with solvents claimed in claims 1c-1f involves the mixing of said materials with the respective solvents using suitable agitation over extended period of time, if necessary, followed by filtration, resulting in a filtrate that comprises the novel non-alcoholic, non-aqueous fragrance materials or fragrance compositions described in the following claims, or
h) wherein the extraction of materials described in claim 1b with solvents claimed in claims 1c-1f involves the mixing under high-energy transfer conditions comprising ultrasound emulsification, rotor-stator emulsification or high-pressure emulsification, followed by centrifugation and refrigeration in order to break said emulsions into two phases, whereby the upper, less polar phase comprises the novel non-alcoholic, non-aqueous fragrance materials or fragrance compositions described in the following claims.

2. Non-alcoholic, non-aqueous fragrance ingredients comprising
a) extracts of raw fragrance materials of botanical, animal, mineral, or synthetic origin, or mixtures thereof, as claimed in claim 1, with solvents or solvent mixtures as claimed in claim 1c-1f, that can be used either directly with out further processing, or concentrated by methods known to the art comprising evaporation or distillation, resulting in products known to the art as “concretes”, which can be further processed by maceration with alcohol resulting in what is known to the art as “absolutes”,
b) artful blends of such extracts as claimed in claim 2a, which may be used in fragrance applications such as the scenting of household products comprising room sprays or detergents, or personal care products such as cremes, lotions, body washes or shampoos, provided the solvents claimed in claim 1c-1f are of cosmetic grade, and
c) fragrance extracts or their respective blends as claimed in claim 2a and 2b, that can be labeled using terminology of the trade comprising “all-natural”, “nature-derived”, “plant based”, or “sustainably produced”, and/or certified as such by one of the certification bodies recognized by the art (ECOCERT, NPA, etc.), provided that the ingredients other than the solvents claimed in 1c-1f are in compliance with those definitions.

3. Non-alcoholic, non-aqueous fragrance compositions or perfumes comprising
a) blends of fragrance extracts as claimed in claim 2, with or without further addition of conventional fragrance materials, wherein the least polar fragrant components are highly enriched in the lipophilic solvent matrix claimed in claims 1c-1f, comprising products with enhanced physical, chemical, olfactory, and/or esthetic characteristics, compared to what prior art can provide;
b) extracts of conventional undiluted fragrance materials comprising what is known to the art as concretes, absolutes, resinoids, prefabricated extracts, and essential oils, or their artful blends, employing the solvents and methodology claimed in claim 1h, instead of the traditional dilution with ethanol, and the subsequent clarification steps used in current practice;
c) compositions as claimed in claim 3b, which—based on the different polarities and affinities of the solvents claimed in claims 1c-1f—have novel olfactory characteristics, when compared to classical alcoholic compositions of the same ingredients, and higher olfactory impact;
d) compositions as claimed in claim 3a, 3b, and 3c, where traditional carrier solvents such as vegetable oils or diluted ethanol have been replaced by the solvents claimed in claims 1c-1f, rendering those compositions entirely lipophilic, and yet volatile;
e) compositions such as claimed in claim 3d, where the respective carrier solvents as claimed in claims 1c-1f are of cosmetic grade, so that they can be either directly applied to the human skin with improved esthetic characteristics over alcohol-based or oil-based compositions, or become the fragrant and emollient part of cosmetic skin- and hair-care formulas;
f) compositions such as claimed in claims 3a-3e, wherein the solvents claimed in claims 1c-1f, together with the majority of the other components in those compositions, are of natural or nature-derived grade, allowing for product labeling privileges of using terminology of the trade comprising “all-natural” or “nature-derived”, and which may be certified as such by one of the certification bodies recognized by the trade (ECOCERT, NPA, etc.).
